(19) United States
(12) Patent Application Publication
(10) Pub. No.: US 2025/0116677 A1
(43) Pub. Date: Apr. 10, 2025

(54) METHOD AND APPARATUS FOR A PREGNANEDIOL URINE ASSAY

(71) Applicant: MFB Fertility, Inc., Erie, CO (US)

(72) Inventor: Amy BECKLEY, Erie, CO (US)

(73) Assignee: MFB Fertility, Inc., Erie, CO (US)

(21) Appl. No.: 18/742,989

(22) Filed: Jun. 13, 2024

Related U.S. Application Data
(63) Continuation of application No. 16/544,554, filed on Aug. 19, 2019, now abandoned.
(60) Provisional application No. 62/272,953, filed on Dec. 30, 2015.

(51) Int. Cl. G01N 33/543; G01N 33/74

(52) U.S. Cl. CPC G01N 33/743; G01N 33/54306; G01N 33/54388

(57) ABSTRACT

An in-home lateral flow assay test strip that can be used to monitor PdG levels in urine, which correlate with progesterone levels in serum. The lateral flow test strip is a competitive assay comprising a sample pad, conjugate pad, membrane strip, backing card, an adsorbent pad, a test line impregnated onto the membrane strip comprised of pregnanediol glucuronide (PdG) conjugated to immunoglobulin G (IgG), bovine serum albumin (BSA), bovine gamma globulin (BGG) or another suitable carrier protein and a control line impregnated onto the membrane strip comprised of anti-mouse antibodies. The conjugate pad is saturated with mouse monoclonal anti-PdG antibodies conjugated to colloidal gold, latex beads, or similar visual dye. The test strip may be encased in a cassette such that only the wicking pad and detection zone are visible to the user.

### RELATED APPLICATIONS

This application is conversion application of U.S. Provisional Patent Application No. 62/720,953, filed on Aug. 22, 2018 and a continuation-in-part application of U.S. Non-provisional patent application Ser. No. 15/900,794, filed Feb. 20, 2018, which is a conversion application of U.S. Provisional Patent Application No. 62/460,307, filed Feb. 17, 2017, the entire contents of said applications are hereby incorporated by reference. This application is also a continuation-in-part of U.S. Non-provisional patent application Ser. No. 15/974,229, filed May 8, 2018, which is a conversion of U.S. Provisional Application No. 62/503,223, filed May 8, 2017, the entire contents of said applications are hereby incorporated by reference. This application is also a continuation-in-part of U.S. Non-provisional patent application Ser. No. 16/381,229, filed Apr. 11, 2019, which is a national stage application of PCT Application No. PCT/US18/68027, filed Dec. 28, 2018, which is a PCT application claiming priority to U.S. Provisional Application No. 62/611,467, filed Dec. 28, 2017, the entire contents of said applications are hereby incorporated by reference.

### FIELD OF THE INVENTION

The present invention relates to the field of hormone diagnostics. More specifically, the present invention relates to urine based lateral flow assays for the detection of progesterone and methods of digital quantification thereof.

### BACKGROUND OF THE INVENTION

The menstrual cycle is controlled by the anterior pituitary gonadotropins follicle-stimulating hormone (FSH) and luteinizing hormone (LH) and the gonadal sex hormones estradiol and progesterone. Estrone-3-glucuronide (E3G) is a principal metabolite of estradiol and the urinary levels of E3G correspond to the serum levels of estradiol. Similarly, pregnanediol-3-glucuronide (PdG) is a principal metabolite of progesterone and the urinary levels of PdG correspond to the serum levels of progesterone, as depicted in FIGS. 2 and 3.

FSH begins the menstrual cycle by stimulating growth of a follicle or follicles. Increased estrogen from a growing follicle triggers a sudden spike in LH. The surge in LH causes the follicle to rupture, which is ovulation. The ruptured follicle then secretes progesterone. Progesterone acts to thicken the uterine wall to prepare for implantation and protect the growing fetus. Once a fertilized embryo has implanted in the uterine wall, human chorionic gonadotropin (hCG) is released and is detectable in urine within a short period of time.

Various different at home urine based hormone tests are currently readily available to consumers to help track the hormones involved in the female menstrual cycle, including tests for FSH, LH and hCG. There are other at home tests that include E3 and LH together to give a digital reading to indicate low, high or peak fertility. However, there remains a need for a urine based at-home test that can provide the consumer information regarding their progesterone levels by detecting and even quantitating PdG.

Moreover, others have unsuccessfully attempted to create a urine-based test to provide information regarding progesterone levels by detecting and quantitating PdG in a context other than in a lab environment, such as for at home for use by a non-expert user. Such attempts have proven fruitless due to inaccuracies associated with the tests with regard to the precision of detection and measurement of PdG. Further, the proper chemistry to enable the creation of such a test remains to be discovered. Specifically, it has yet to have been discovered how to create a progesterone test visible to the naked eye despite years of effort. Currently, progesterone tests remain limited to use within a lab environment. Colorimetric lab-grade electronic readers are used to detect differences in color otherwise imperceptible to the naked eye. Such lab-based tests determine concentrations to a high accuracy, often with the assistance of lasers. To create a test allowing one to visually review the results with the naked eye and without the assistance of lab-grade equipment, an alternative solution is needed. Previous attempts to create a lateral flow assay for detecting progesterone metabolites in urine, including the inventive matter disclosed in U.S. Pat. No. 6,924,153 granted on Aug. 2, 2005, the inventive matter disclosed in United Kingdom Patent Application Publication No. GB 2,204,398 A as published on Nov. 9, 1988, and similar prior art items, were unsuccessful due to the technical difficulties and inappropriate selection of component antibodies (namely the selection of component antibodies of improper isotypes) and type of carrier proteins. In certain cases, such difficulties also were associated with the development antigen and antibody chemistries of such ratios, component parts and/or elements to specifically produce visual results readable to the naked eye. Other prior art matter, for instance the subject matter disclosed in PCT/FR2016/050506 published on Mar. 4, 2016, only discloses Bovine Serum Albumin (BSA) as the carrier protein, which is a commonly used carrier protein and inadequate for usage in a urine-based progesterone or PdG testing solution intended to display results visible and discernable to the naked eye. Among other challenges associated with the solution disclosed in PCT/FR2016/050506, its disclosure of BSA as the carrier protein results in a testing solution lacking the ability to adequately bind to colloidal gold, thereby resulting in a test delivering results that are problematically imperceptible to the naked eye to the necessary usable perception level. Moreover, these and other prior art solutions have failed to produce a product that reliably and reproducibly produced enough color intensity to deliver clear and easily interpreted test results to users with minimal training and a lack of specialized equipment. Therefore, a need remains for a lateral flow assay for detecting progesterone metabolites in urine that reliably and reproducibly delivers enough color intensity to portray clear and easily interpreted test results to users with minimal training and a lack of specialized equipment.

Prior art solutions are associated with challenges stemming from problematic antibody selection and incorporation, often due to the selection and incorporation of improperly chosen antibodies and antibody isotypes. A problem associated with prior art solutions is that the specifically chosen antibodies with such solutions are undesirably cross-reactive. In certain cases, chosen antibodies have suboptimal affinities for the application of a PdG test. The chosen antibodies in prior art solutions are outside of a desired detection range. For instance, the chosen antibodies result in a test that is not sensitive enough to allow a user to distinguish a positive and negative result. Sensitivity in such context may derive from suboptimal levels of affinity, avinity and specificity. In prior art tests where suboptimal sensitivity results from suboptimal specificity, the chosen antibody having a particular antibody isotype binds on items other than a PdG target. A problem with prior art tests having a particular suboptimal combination antibody, antibody isotype and/or carrier protein, is that the antibody and the conjugate do not bind with the precision necessary to produce a viable, reproducible test result useful to detect the presence of PdG.

### BRIEF SUMMARY OF THE INVENTION

It is an object of the present invention to address several challenges in previous attempts to create a urine-based PdG tests. The present invention is an in-home lateral flow assay test strip that can be used to monitor PdG levels in urine, which correlate with progesterone levels in serum (hereinafter “PdG test strip”). The present inventor has arrived at a specifically configured combination of elements to create a preferred embodiment of a membrane strip comprised of 1) anti-pregnanediol glucuronide (PdG) antibodies of a specific isotype, IgG2b in the preferred embodiment, conjugated to visual label, and 2) PdG hormone conjugated specifically to bovine gamma globulin (BGG).

In one embodiment, a urine sample is applied the wicking pad (or a well connected to the wicking pad) and by lateral flow, the sample is allowed to permeate through the strip material into or through the various detection zones in which the strip is coated with specific binding partners. PdG from the urine sample becomes bound within the detection zone and the extent to which analyte becomes bound can be determined by labeled secondary reagents such as colored latex beads or colloidal gold. The color intensity in each detection zone is directly correlated with the concentration of the analyte in the sample. The PdG test strip is single-use and disposable after reading the results.

The PdG test strip described above may be used in a variety of different scenarios. Such scenarios include, utilization as part of a digital method of quantifying and tracking PdG levels throughout the menstrual cycle. The user will collect urine samples on different days of her menstrual cycle and use a PdG test strip with each urine. Moreover, the present invention is useful to identify the fertile and non-fertile phase of a women's menstrual cycle. Furthermore, if conception occurs, progesterone and PdG levels remain elevated throughout the duration of the pregnancy, trackable by use of one embodiment of the invention. The PdG test strip has other very useful hormone monitoring uses. For example, the ovaries produce progesterone in large concentrations after ovulation has taken place. Therefore, lack of progesterone production over several weeks or months could indicate the start of menopause. Low amounts of progesterone could indicate an ovarian dysfunction such as failed ovulation attempt, luteinized unruptured follicle (LUF), or poor ovulation. Low or abnormal progesterone levels could also indicate a luteal phase defect. Additionally, in certain cancers such as breast, ovarian, and uterine, cancer cells are responsive or can secrete progesterone.

One embodiment of the invention is directed to a pregnanediol urine assay having specific reagent combinations, said specific reagent combinations uniquely enabling a strong enough interaction in the testing zone to allow for visual, naked eye inspection of the test results.

### BRIEF DESCRIPTION OF THE DRAWINGS

FIG. 1 is perspective view of the lateral flow assay design of the present invention.

FIG. 2 is a chart displaying the average amount of PdG in urine if conception occurs.

FIG. 3 is a chart displaying the average amount of PdG in urine if conception does not occur.

FIG. 4A shows exemplary control and test line intensities for a negative test result.

FIG. 4B shows exemplary control and test line intensities for a positive test result.

FIG. 4C shows exemplary control and test line intensities for an invalid result.

FIG. 5 is a chart showing increase in color intensity of the test line as PdG concentration increases.

### DETAILED DESCRIPTION

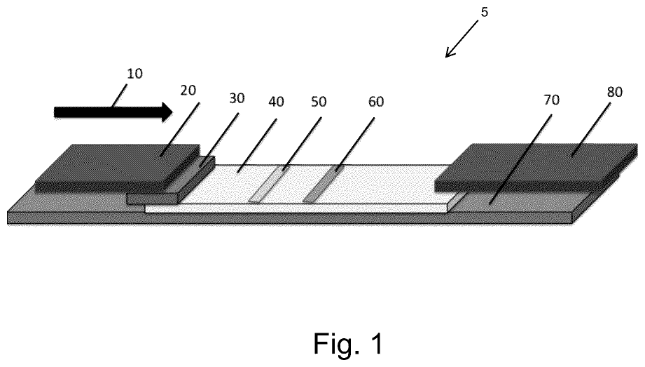

Referring now to FIG. 1, a test strip 5 of the present invention is shown wherein an end with a sample pad 20 is dipped into a urine sample such that the urine sample flows up the strip 5 in a direction depicted by an arrow 10 and is stopped by an adsorbent pad 80 at an end opposite the sample pad 20. The sample pad 20 is readily available from various supplies, such as SureWick® Pad Materials from Millipore Sigma. The strip 5 includes a conjugate pad 30 that can be a glass fiber conjugate pad saturated with colloidal gold, colored latex beads or other visual dye particles that are conjugated to anti-pregnanediol glucuronide mouse monoclonal antibodies. A membrane 40 of the strip 5 can be a nitrocellulose membrane with pore size between 3 to 20 μm. A test line 50 and a control line 60 are impregnated on the membrane 40. The membrane 40 is supported by a backing card 70.

In the one embodiment, the test line 50 is comprised of pregnanediol glucuronide (PdG) (cas #1852-49-9) conjugated to bovine gamma globulin (BGG); however, the PdG may also be conjugated to other suitable carrier proteins such as immunoglobulin G (IgG), bovine serum albumin (BSA). In the preferred embodiment, the conjugate pad 30 is saturated with mouse monoclonal anti-PdG antibodies conjugated to colloidal gold, latex beads, or similar visual dye. The antibody should not be reactive with the following hormones: (1) testosterone; (2) testosterone glucuronide; (3) aldosterone; (4) cortisol; (5) corticosterone; (6) 11-deoxycortisol; (7) 21-deoxycortisol; (8) dehydroepiandrosterone; (9) dehydroepiandrosterone 3-sulfate sodium salt; (10) dexamethasone; (11) 17a-hydroxyprogesterone and (12) progesterone.

The PdG test strip described herein does not give a false positive when Hcg<2000 mlU/ml; Lh<50 mlU/ml; Estrone-3 glucuronide<200 ng/ml; and FSH<30 mlU/ml. These values are maximum hormone levels observed in women who are either not pregnant or very early in pregnancy.

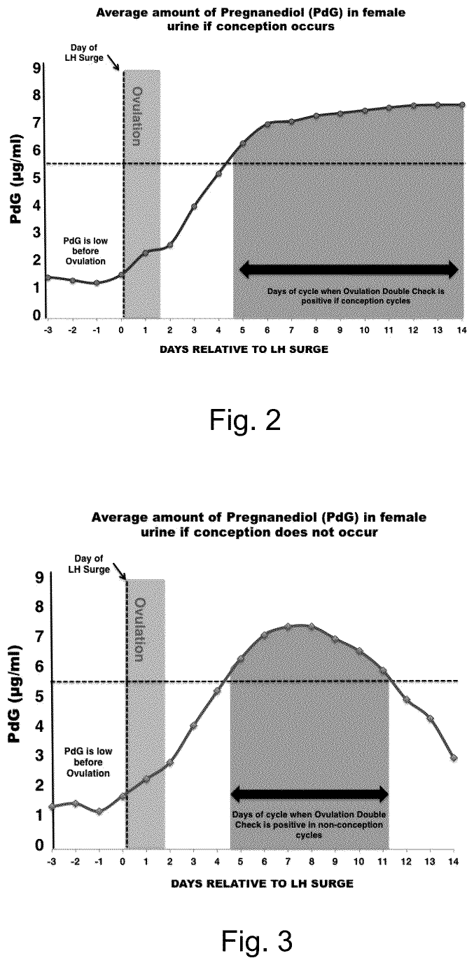

FIG. 2 is a chart displaying the average amount of PdG in urine if conception occurs. As can be seen, PdG in urine is low before ovulation. After ovulation, PdG increase as the uterus prepares for implantation. If conception does not occur, PdG levels plummet indicating that serum progesterone levels have also decreases accordingly. Without progesterone, the uterine wall can't be maintained, menstruation begins, and the menstrual cycle starts over. As such, the PdG test strip 5 of the present invention is a useful test to determine that a woman has already ovulated and is no longer fertile until at least after menstruation has started.

FIG. 3 is a chart displaying the average amount of PdG in urine if conception does not occur. If conception does occur, PdG levels remain high so the uterus can support the growing fetus. Adequate progesterone is essential for both getting pregnant and maintaining a healthy pregnancy. As such, the PdG test strip 5 of the present invention is a useful test to monitor PdG in urine during early pregnancy as it corresponds with serum progesterone levels. If levels are low after ovulation or start to decrease after pregnancy is confirmed by hCG testing, a woman can relay this information to their medical professional and possibly start progesterone supplementation to help them either get pregnant and avoid miscarriage caused by low progesterone levels.

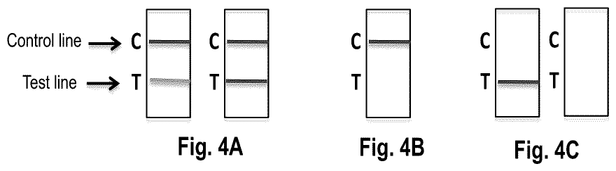

The PdG test strip 5 has a threshold value of 5-6 ug/ml PdG such that urine samples with <5 μg/ml PdG will show two lines as shown in FIG. 4A. FIG. 4B shows that urine samples with >5 μg/ml will only show one line, which indicates a positive result. FIG. 4C depicts a scenario where the control line does not appear. In this case, the test results are invalid.

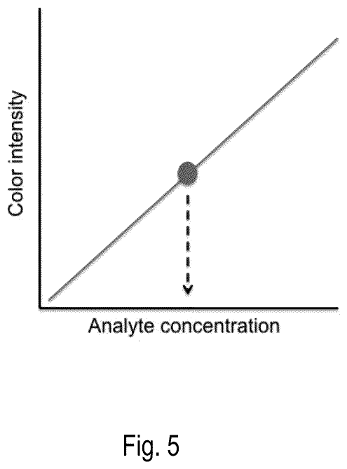

FIG. 5 is a chart showing the relation between color intensity of the test line 50 as PdG concentration increases. In light of this, the PdG test strip 5 can be used as part of a system to monitor/track hormone levels throughout the menstrual cycle and during early pregnancy. The PdG test strip 5 may be used alone as shown in FIG. 1 or within a cassette that is known in the prior art to house the PdG test strip 5. The PdG test strip 5 (or the cassette) may have a unique identifier such as lot number, barcode, or QR code. Samples of known PdG concentrations are applied to the test strip 5 and each concentration yields a test line of a different color intensity. The color intensities are plotted on a graph to create a standard curve. All unknown samples (pictured as a dot in FIG. 5) that fall between the lowest and highest standard curve intensity will be assigned a concentration along the standard curve line.

The present inventor has discovered a unique combination of specific elements to allow for the detection of pregnanediol glucuronide (PdG) formulated such as to enable the creation of a pregnanediol urine test. In the preferred embodiment of the invention, Bovine Gamma Globulin (BGG) is conjugated with PdG and combined with a mouse anti-PdG antibody of IgG2b isotype binding partner. In an alternative embodiment of the invention Bovine Gamma Globulin (BGG) conjugated to PdG is combined with a mouse anti-PdG antibody of IgG1, IgG1 Kappa, IgG2a or IgG2c isotype. The present inventor has recognized that such a specific combination uniquely allows for colloidal gold to be conjugated to the anti-PdG antibody of one of the specific isotypes mentioned above, and for the colloidal gold conjugated anti-PdG antibody to interact with the PdG-BGG conjugate. Other combinations have been attempted, and have failed to allow the colloidal gold to function to produce the color needed to allow the test results to be viewable visually by the naked and untrained (layperson) eye. The present inventor has noted that the utilization of BGG conjugated to PdG allows for anti-PdG antibody, specifically of the IgG2b isotype, to bind in such a manner that colloidal gold is carried at a concentration sufficient for naked eye visualization. The present inventor has recognized the benefit associated with embodiments of the invention that a PdG test may be producible allowing the results to be visually interpreted with the naked eye.

The preferred embodiment of the present invention comprises a testing system to detect the presence of PdG optimized for visual detection by a layperson's, or non-expert's, naked eye utilizing the embodiment in other than a laboratory context. The present inventor has recognized that in embodiments of the invention, the combination of mouse anti-PdG IgG1, IgG2a, IgG2b, and/or the IgG2c antibody conjugated to a visual label, such as colloidal gold and/or latex beads, and PdG conjugated to BGG carrier protein create sufficient binding partners. Resultantly, the preferred embodiment of the invention comprises a visual test readable by the untrained eye in a context outside of a laboratory environment, as depicted in FIGS. 4A-4C.

The preferred embodiment of the invention relies on the certain reagents being able to interact with other reagents to produce color in the test zone of the membrane. Specifically, in the absence of PdG hormone in the urine sample, the following reagents must interact in order for the test results to be useful. First, in the preferred embodiment, colloidal gold must be conjugated to the anti-PdG antibody (in the preferred embodiment, anti-PdG antibody having the IgG2b isotype). In alternative embodiments, as a replacement for colloidial gold in other embodiments described herein, an alternative visual dye such as latex beads may be utilized to a similar effect. Further, in embodiments of the invention, the colloidal gold conjugated anti-PdG antibody must interact with the PdG-BGG conjugate. Moreover, the PdG-BGG must bind the nitrocellulose membrane. The present inventor has recognized that for these embodiments to function as intended, these interactions between and among the colloidal gold conjugated anti-PdG antibody and the PdG-BGG conjugate must be strong enough and stable enough to form and stay bound during urine sample application and lateral flow of urine across the reaction zone to solve the problems faced by the suboptimal prior art mechanisms.

The present inventor has discovered that since PdG is a small hormone metabolite, in order to strongly bind to the surface of a membrane, PdG requires a strong carrier protein. However, the present inventor has discovered that, for the preferred embodiment of the invention to function as intended, not only does the strong carrier protein need to bind the nitrocellulose membrane, but the strong carrier protein also needs to bind the PdG and present it to the anti-PdG antibody. Prior to the embodiments of the invention as disclosed herein, other attempts in the prior art have failed to include an optimal combination of a strong carrier protein able to bind the PdG and present it to the anti-PdG antibody.

In embodiments of the invention, one carrier protein is conjugated to four or more PdG molecules. In the preferred embodiment, the one carrier protein is conjugated to no more than eight PdG molecules. The present inventor has discovered that such a ratio allows for the colloidal gold conjugated anti-PdG antibody to bind with both enough affinity and avidity to produce a bright enough color in the test reaction zone for typical users to distinguish visually. The present inventor has discovered the specific property of BGG enabling such combination. In embodiments of the invention, as BGG exhibits the optimal number of active sites optimally spaced, the inclusion of BGG results in a lesser amount of steric hindrance, and therefore embodiments of the invention are enabled to receive and bind PdG at sufficient ratios. Therefore, BGG is essential for the preferred embodiment of the invention to function as intended. In the preferred embodiment of the invention, therefore, PdG is conjugated to BGG.

Another critical component of the preferred embodiment of invention is the specifically chosen anti-PdG antibody. In order for the preferred embodiment of the invention to function as intended, the specifically chosen anti-PdG antibody needs to be monoclonal, due to the nature of the PdG antigen presentation on the BGG conjugate. In order for the embodiments of the invention to function as intended, the specifically chosen anti-PdG antibody must incorporate one of the following isotypes: IgG1, IgG2a, IgG2b, or IgG2c. The present inventor has discovered that isotypes other than IgG1, IgG2a, IgG2b, or IgG2c, including but not limited to IgM, IgS, and IgE anti-PdG antibody isotypes, remain unable to effectively bind the colloidal gold (or other visual label) and produce a strong enough color signal on the reaction zone due to their size and structure and are therefore excluded from the preferred embodiment of the invention. Since the colloidal gold must bind the Ig region of the anti-PdG antibody, the present inventor has discovered that the IgG1, IgG2a, IgG2b, and IgG2c isotypes of the anti-PdG antibody sufficiently bind colloidal gold and are therefore incorporated into embodiments of the invention. As a result, the IgG1, IgG2a, IgG2b and IgG2c isotypes of the anti-PdG antibody therefore produce the strongest color. In the preferred embodiment of the invention, the IgG2b isotype is included in the invention, as the present inventor has recognized that the IgG2b isotype performs slightly better when producing color. Therefore, the preferred embodiment of the invention incorporates the IgG2b isotype of the anti-PdG antibody. Alternative embodiments of the invention incorporate the IgG2a, IgG2c or IgG1 isotypes of the anti-PdG antibody.

In embodiments of the invention, therefore, the conjugate striped on the membrane in the test zone area is PdG-BGG and the anti-PdG antibody must be a monoclonal anti-PdG antibody of one of the following isotypes: IgG1, IgG2a, IgG2b, or IgG2c.

Specific method steps more specifically broken out, include:

Selecting a PdG antibody of a specific isotype. In the preferred embodiment, the specific PdG antibody chosen is anti-PdG antibody of the IgG2b isotype. In alternative embodiments, the specific anti-PdG antibody chosen is one of the anti-PdG antibodies of either the IgG1, IgG2a, or IgG2c isotypes. Conjugating the selected anti-PdG antibody to visual dye. In the preferred embodiment, the visual dye consists of colloidal gold of a size between 20-100 nM in diameter. In alternative embodiments, the visual dye comprises colloidal gold or colored latex beads.

Saturating the conjugate pad with the visually labeled anti-PdG antibody. In embodiments of the invention, the visual labelization takes place as a result of conjugating an anti-PdG antibody to a visual dye, such as colloidal gold.

Conjugating PdG to BGG carrier protein at a ratio of four (4) or more units of PdG per 1 unit of BGG.

Impregnating PdG conjugated to BGG onto a competitive assay lateral flow test strip membrane at a concentration of 0.5-2 mg/ml in the test zone area, and impregnating anti-mouse antibodies into the control zone area.

In the preferred embodiment, a viable PdG test results from the above method. The preferred method of usage of the PdG test in an embodiment of the invention follows: First, dipping assay lateral flow strip into urine sample. No additional buffers or sample treatment are needed. Second, reading the results displayed on the assay with the naked eye, following 1 to 10 minutes of waiting. Third, interpreting the results as either positive or negative. A negative result is displayed as the presence of two dark colored lines, one in the control zone area, and one in the test zone area. A positive result is displayed as the absence of a line in the test zone area and the presence of a single line in the control zone area.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the method (and components of the individual operating components of the method) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections might be present in a practical device. Moreover, no item or component is essential to the practice of the invention unless the element is specifically described as “essential” or “critical”. Further, the steps of any method described herein may be utilized in a different order in alternative embodiments of the invention. Numerous modifications and adaptations will be readily apparent to those skilled in this art without departing from the spirit and scope of the present invention.

What is claimed is:

1. A test strip assay, comprising:
a conjugate pad saturated with mouse monoclonal anti-pregnanediol glucuronide (anti-PdG) antibodies of IgG2b isotype;
a membrane strip;
a test line impregnated onto the membrane strip comprised of pregnanediol glucuronide (PdG) conjugated to bovine gamma globulin (BGG); and
a control line impregnated onto the membrane strip comprised of anti-mouse antibodies.

2. The test strip assay of claim 1, the mouse monoclonal anti-PdG antibodies of IgG2b isotype conjugated to a visual dye.

3. The test strip assay of claim 1, further comprising a sample pad.

4. The test strip assay of claim 1, further comprising a backing card.

5. The test strip assay of claim 1, further comprising an adsorbent pad.

6. A test strip assay, comprising:
a conjugate pad saturated with mouse monoclonal anti-PdG antibodies of isotypes selected from the following group: IgG2a isotype, IgG2c isotype, or IgG1 isotype;
a membrane strip;
a test line impregnated onto the membrane strip comprised of pregnanediol glucuronide (PdG) conjugated to bovine gamma globulin (BGG); and
a control line impregnated onto the membrane strip comprised of anti-mouse antibodies.

7. The test strip assay of claim 6, the mouse monoclonal anti-PdG antibodies of isotypes selected from the following group: IgG2a isotype, IgG2c isotype, or IgG1 isotype; conjugated to a visual dye.

8. The test strip assay of claim 6, further comprising a sample pad.

9. The test strip assay of claim 6, further comprising a backing card.

10. The test strip assay of claim 6, further comprising an adsorbent pad.

11. A method for creating an urine based ovulation test by combining a pregnanediol (PdG) antibody with Bovine Gamma Globulin (BGG), comprising:
selecting a PdG antibody of a specific isotype;
conjugating the selected anti-PdG antibody to a visual dye;
saturating a conjugate pad with the visually labeled anti-PdG antibody;
conjugating PdG to BGG at a ratio greater than or equal to 4 units of PdG per 1 unit of BGG;
impregnating a PdG conjugated to BGG onto a competitive assay lateral flow test strip membrane at a concentration within the range of 0.5-2 mg/ml in a control zone area; and
impregnating anti-mouse antibodies into the control zone area.

12. The method of claim 11, the anti-PdG antibody having the IgG2b isotype.

13. The method of claim 11, the anti-PdG antibody having the IgG2a isotype.

14. The method of claim 11, the anti-PdG antibody having the IgG2c isotype.

15. The method of claim 11, the anti-PdG antibody having the IgG1 isotype.
